(19) United States
(12) Patent Application Publication
(10) Pub. No.: US 2019/0321431 A1
(43) Pub. Date: Oct. 24, 2019

(54) PREPARATION AND APPLICATION OF Syzygium zeylanicum L. EXTRACT

(71) Applicant: Tamkang University, New Taipei City (TW)

(72) Inventors: SAN-LANG WANG, New Taipei City (TW); VAN-BON NGUYEN, Buon Ma Thuot City (VN); ANH-DZUNG NGUYEN, Buon Ma Thuot City (VN); QUANG-VINH NGUYEN, Buon Ma Thuot City (VN)

(21) Appl. No.: 16/207,713

(22) Filed: Dec. 3, 2018

(30) Foreign Application Priority Data
Apr. 24, 2018 (TW) 107113911

(51) Int. Cl. A61K 36/61; A61K 9/00; A61P 3/10; A61P 3/04

(52) U.S. Cl. CPC A61K 36/61; A61K 9/0053; A61P 3/10; A61K 2236/51; A61K 2236/15; A61K 2236/331; A61P 3/04

(57) ABSTRACT

A preparation and application of Syzygium zeylanicum L. extract, which is produced by solvent extraction. The extract can reduce α-glucosidases and α-amylases after administration in vivo and does not cause side effects, further can control the abnormal performance of blood sugar in diabetic patients after meals.

### BACKGROUND OF THE INVENTION

### 1. Field of the Invention

The present disclosure relates to a preparation and application of the extract of Syzygium zeylanicum L., and more particularly to the application of alpha-glucosidase and alpha-amylase reduction by oral administration after the meal.

### 2. Description of the Prior Arts

There are dozens of enzymes in the human digestive system to digest or decompose food. After digestion, the food will release the nutrients and calories for absorption. The “enzymes inhibitors” could inhibit enzymes, so that the food cannot be decomposed and not be absorbed by the body. The mechanism of commercially starch inhibitors in the diet foods is also blocking the digestion and absorption of starch and reducing calorie intake.

After ingesting carbohydrate, the human body will decompose the polysaccharide into oligosaccharide molecules through the α-amylase secreted by saliva, gastric juice or pancreas, and then the oligosaccharide molecules will enter the small intestine. The α-glucosidase from the epithelial cells and the digestive enzymes in the gastrointestinal tract will decompose the oligosaccharide molecules into glucose that can be absorbed by the body, and the blood glucose concentration in the body is highest. Therefore, α-glucosidase inhibitors and α-amylase inhibitors can significantly reduce the digestion and absorption of carbohydrates, and reduce the increase in postprandial blood glucose levels in non-insulin-dependent diabetic patients.

Type 2 diabetes is one of the most important chronic diseases in the world, and the annual prevalence rate is increasing year by year. Nowadays, oral hypoglycemic agents as the main treatment policy of type 2 diabetes in western medicine. Hypoglycemic agents can be roughly classified into eight categories such as sulfonylurea, biguanide, thiazolidinedione, α-glucosidase inhibitor, dipeptidyl peptidase inhibitor (DPP-4 inhibitor), incretin GLP-1 agent (glucagon-likepeptide-1 agent), sodium-glucose cotransporter 2 inhibitor (SGLT-2 inhibitor), and insulin.

Acarbose is an oligosaccharide that can inhibit the digestive enzymes in the small intestinal brush border cells after administration. This can slow down the digestion of carbohydrates, thereby delaying the glucose absorption and alleviating postprandial hyperglycemia and hyperinsulinemia in diabetic patients. Acarbose can avoid postprandial hyperglycemia and achieve a stable postprandial blood glucose control, so it is the most commonly used inhibitor. The digestive enzymes include: α-glucosidases and α-amylase. However, acarbose will delay the food digestion and absorption, and cause some undigested food to enter the colon directly. Some undigested food will be fermented by intestinal bacteria, and further leading to gastrointestinal symptoms such as flatulence and diarrhea. Therefore, a new pharmaceutical is needed for inhibiting digestive enzymes in small intestinal brush border cells in the related art.

Syzygium zeylanicum L. is a plant of the genus Myrtaceae, which is mainly grown in India to Malaysia and has medical functions for treating rheumatism and syphilis. Macrocyclic ellagitannin, zeylaniin and other substances with strong anti-oxidation function can be extracted from the leaves. Studies have also pointed out that Syzygium zeylanicum L. has good antibacterial effect and various nutrients (Shilpa, K. J., & Krishnakumar, G. (2015). Nutritional, fermentation and pharmacological studies of Syzygium caryophyllatum (L.) Alston and Syzygium zeylanicum (L.) DC fruits. Cogent Food & Agriculture, 1(1), 1018694.).

### SUMMARY OF THE INVENTION

The object of the present invention is to provide an inhibitor extracted from natural Syzygium zeylanicum L. to inhibit digestive enzymes in small intestinal brush border cells, and further provide an oral inhibitor for the user after meal to reduce α-glucosidases and α-amylase to achieve good blood sugar control. Since the oral inhibitor of the present invention is extracted from a natural product, it does not cause side effects after administration, and can be used with an existing inhibitor to further effectively control blood glucose of a diabetic patient.

To achieve the above objective, the extraction steps of Syzygium zeylanicum L. extract of the present invention include:

Step 1: grinding a portion of Syzygium zeylanicum L. at room temperature into a powder;

Step 2: mixing the powder and a solvent at a ratio of 1:10 by volume, and shaking at room temperature for 24 hours, and producing a mixture;

Step 3: filtering the mixture of the step 2, and producing a residue and an extract;

Step 4: mixing the residue of the step 3 and the mixture of the step 2 by 20 times volume of the powder of the step 1, and then shaking at room temperature for 24 hours;

Step 5: repeat at least once of the steps 3 and 4, and collecting the extract; Step 6: vacuum drying the extract from step 5 at 60° C. to obtain Syzygium zeylanicum L. extract and storing at −30° C.

### BRIEF DESCRIPTION OF THE DRAWINGS

FIG. 1 is a schematic view of the alpha-glucosidase with IC₅₀ inhibited by the extract obtained by the solvents of the example 1 of the present invention.

FIG. 2 is a pH-stability test of the Syzygium zeylanicum L. extract of the example 1 of the present invention.

FIG. 3 is a temperature stability test of the Syzygium zeylanicum L. extract of the example 1 of the present invention.

FIG. 4 is a schematic view showing the inhibiting effect on blood glucose by Syzygium zeylanicum L. extract to 50 mg/kg mice according of the example 4 of the present invention.

FIG. 5 is a schematic view showing the inhibiting effect on blood glucose by Syzygium zeylanicum L. extract to 100 mg/kg mice according of the example 4 of the present invention.

FIG. 6 is a schematic view showing the inhibiting effect on blood glucose by Syzygium zeylanicum L. extract to 200 mg/kg mice according of the example 4 of the present invention.

FIG. 7 is a comparing view of the inhibition of alpha-glucosidase of the example 5.

FIG. 8 is a comparison view of the inhibition of alpha-glucosidase with IC₅₀ of the example 5.

### DETAILED DESCRIPTION OF THE PREFERRED EMBODIMENTS

### Examples

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1 Extracting Syzygium zeylanicum L. Extract

Step 1: grinding a portion of Syzygium zeylanicum L. at room temperature into a powder;

Step 2: mixing the powder and a solvent at a ratio of 1:10 by volume, and shaking at room temperature for 24 hours, and producing a mixture; Step 3: filtering the mixture of the step 2, and producing a residue and an extract;

Step 4: mixing the residue of the step 3 and the solvent of the step 2 by 20 times volume of the powder of the step 1, and then shaking at room temperature for 24 hours;

Step 5: repeat at least once of the steps 3 and 4, and collecting the extract;

Step 6: vacuum drying the extract from step 5 at 60° C. to obtain Syzygium zeylanicum L. extract and storing at −30° C.

In the step 1, the portion of the Syzygium zeylanicum L. includes bark, fruit, and leaves; preferably, the effect of the bark is best.

In the step 2, the solvent includes ionic water, butanol, ethyl acetate, methanol or ethanol.

### Example 2 Enzyme Activity Inhibition Test

The test steps of this example are as follows. Firstly, the Syzygium zeylanicum L. extract was mixed a buffer (potassium phosphate buffer) with 1:9 volume to produce a Syzygium zeylanicum L. extract solution. The Syzygium zeylanicum L. extract solution and alpha-glucosidase (enzyme) were mixed into the buffer at a volume ratio of 1:1. In this example, 15 μl extract solution and the enzyme were separately mixed into 100 μl buffer at 37° C. for 20 minutes. Then, 50 μl matrix p-nitrophenyl glucopyranoside (pNPG) was added at 37° C. for 40 minutes; then 100 μl, 1 mol/L of Na₂CO₃ was added to stop the reaction. Finally, the absorbance of OD 410 nm was measured, and calculated the inhibition activity of alpha-glucosidase. The above experimental steps was the subsequent enzyme activity test method of the present invention, and only different conditions, such as extraction solvent, reaction temperature or pH were changed.

Extract Activity Test of Extract Obtained by Different Extraction Solvents

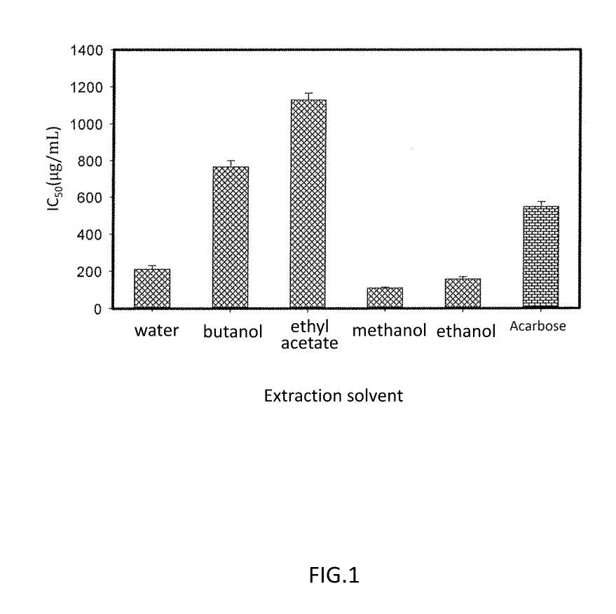

As shown in FIG. 1, Syzygium zeylanicum L. was extracted by different solvents, and performed enzyme activity inhibition test respectively. The Syzygium zeylanicum L. was extracted with ethyl acetate has the highest efficiency IC₅₀ value, and was higher than the conventional acarbose. Thus, ethyl acetate as the solvent for extraction in subsequent examples of the present invention.

Extract Stability Test—pH

Firstly, 0.1 mL Syzygium zeylanicum L. extract (concentration of 1000 μg/mL) was added to a 0.9 mL buffer solution to produce a Syzygium zeylanicum L. extract solution at 37° C. for 30 minutes, wherein the pH of the buffer solution was 2 to 13 respectively, After 30 minutes, the pH was adjusted back to neutral with buffer solution to avoid the damage of the enzyme activity. After that, 15 μl Syzygium zeylanicum L. extract solution and the same volume enzyme were mixed into 100 μl buffer solution for enzyme activity inhibition test.

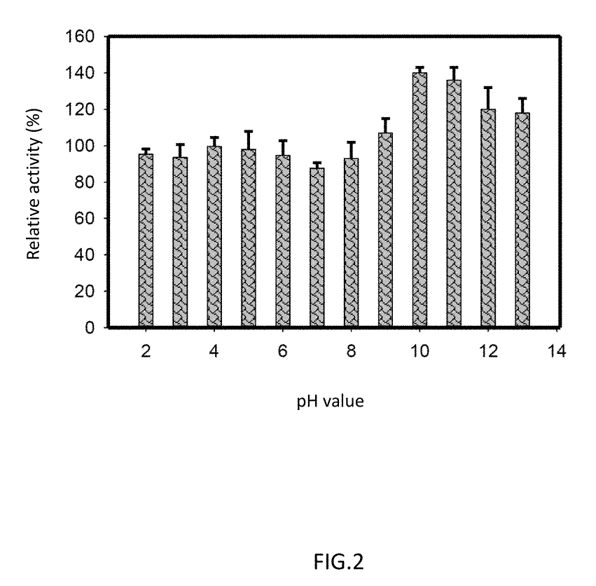

As shown in FIG. 2, when the pH value was from pH 2 to pH 8, the extract of the present invention has good inhibitory activity, and the activity of the extract of the present invention relative to pH<9 has improved while the buffer solution>9.

Extract Stability Test—Temperature

First, the Syzygium zeylanicum L. extract solution was placed into 40° C., 50° C., 60° C., 70° C., 80° C., 90° C. and 100° C. respectively for 30 minutes. After that, the reaction temperature was backed to 37° C. or room temperature to avoid high temperature damage to the enzyme activity, and then 15 μl Syzygium zeylanicum L. extract solution and the same volume of enzyme were mixed into 100 μl buffer solution for enzyme inhibition test.

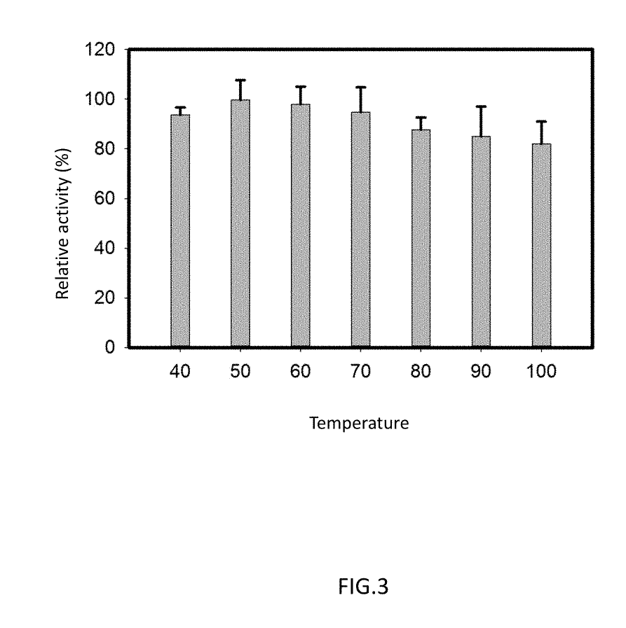

As shown in FIG. 3, the Syzygium zeylanicum L. extract of the present invention has good inhibition of alpha-glucosidase activity after high temperature reaction.

Therefore, the Syzygium zeylanicum L. extract of the present invention has good stability in pH and temperature variation.

### Example 3 Comparison of the Extract of the Present Invention and Acarbose

The enzyme activity inhibition test of Example 2 was used in this example. There were two groups for testing the inhibition effects of different enzymes and calculating the inhibition concentration (IC₅₀) of the enzymes, such as Syzygium zeylanicum L. extract of the present invention and acarbose. The enzymes were S. cerevisiae alpha-glucosidase, rat alpha-glucosidase, B. stearothermophilus alpha-glucoside), porcine pancreatic alpha-amylase, B. subtilis alpha-amylase, and human saliva alpha-amylase.

The results were shown in Table 1, the extract of the present invention had a good inhibitory effect on mammalian alpha-glucosidase. The extract of the present invention could widely inhibit the alpha-starch, and can also inhibit alpha-amylase produced by human saliva.

TABLE 1
Comparison of the efficacy of the extract of the present invention and
acarbose

| | |
|---|---|
| Inhibition of SZL | Inhibition of Acarbose |

| | | Maximum | | Maximum |
|---|---|---|---|---|
| | | Inhibitory | | Inhibitory |
| | IC₅₀ | Activity | IC₅₀ | Activity |
| Enzyme | (μg/mL) | (%) | (μg/mL) | (%) |
| S. cerevisiae alpha- | 0.18 ± 0.01 | 93 ± 2.2 | 1495 ± 10 | 64 ± 2.3 |
| glucosidase | | | | |
| Rat alpha-glucosidase | 109 ± 6.1 | 100 ± 4.1 | 547 ± 2.5 | 84 ± 3.4 |
| B. stearothermophilus | 0.31 ± 0.07 | 98 ± 1.6 | 0.022 ± 0.001 | 100 ± 2.2 |
| alpha-glucoside | | | | |
| Porcine pancreatic alpha- | 3.7 ± 0.21 | 99.2 ± 1.8 | 5.2 ± 0.09 | 91 ± 2.5 |
| amylase | | | | |
| B. subtilis a-amylase | 12.6 ± 1.11 | 93 ± 2.1 | ND | ND |
| Human Saliva alpha-amylase | 1.93 ± 0.02 | 96 ± 2.3 | ND | ND |

ND: non detectable

It could be seen from Table 1 that the extract of the present invention can inhibit alpha-amylase by blocking the digestion and absorption of starch and reducing the caloric intake of starch, especially salivary amylase. Thus, the extract of the present invention had an inhibitory effect to alpha-amylase and blood glucose reduction, and also had weight loss effect.

### Example 4 Animal Experiment

In this example, an animal experiment was conducted by Syzygium zeylanicum L. extract of the present invention for observing postprandial blood glucose suppression in mice. The animals used in this example were ICR mice and were divided into two groups as follow.

Experimental group: the mice were fasted for 16 hours, and then three kinds of dried extracts of the present invention were dissolved in distilled water and respectively administered to mice at a concentration of 50 mg/Kg, 100 mg/kg, 200 mg/kg, wherein “kg” was the body weight of mice. After 20 minutes of oral administration, mice were fed 3 g/kg sucrose. After 0.5 hour, 1 hour, 1.5 hour and 2 hours later, blood of the mice were drawn separately for detecting the blood glucose level.

Control group: the mice were fasted for 16 hours, and then the same volume of distilled water as the experimental group was fed for 20 minutes. After 20 minutes of oral administration, mice were fed 3 g/kg sucrose. After 0.5 hour, 1 hour, 1.5 hour and 2 hours later, blood of the mice were drawn separately for detecting the blood glucose level.

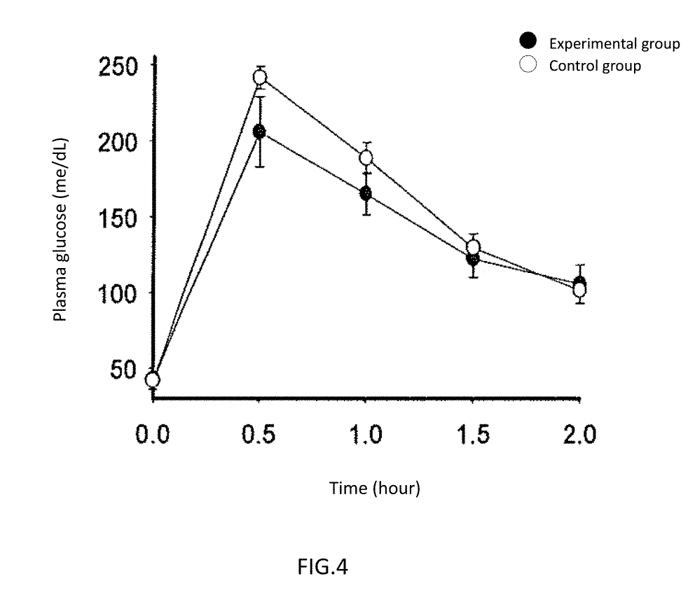

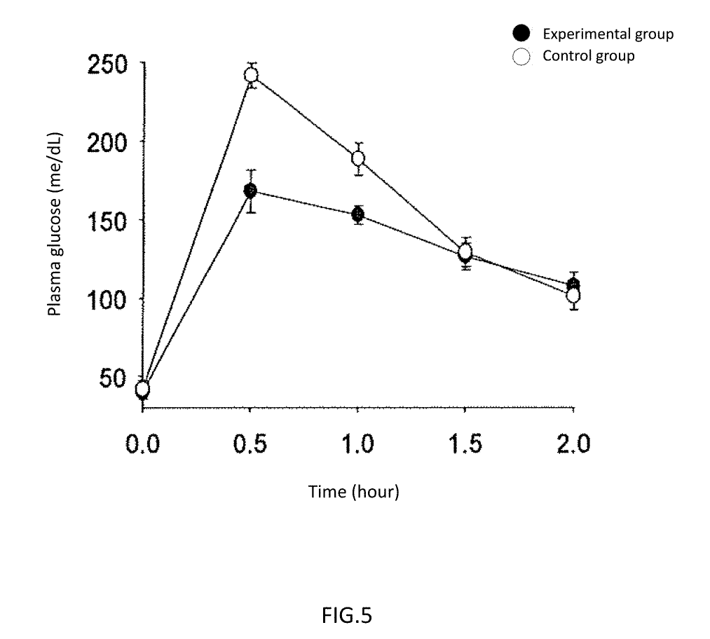

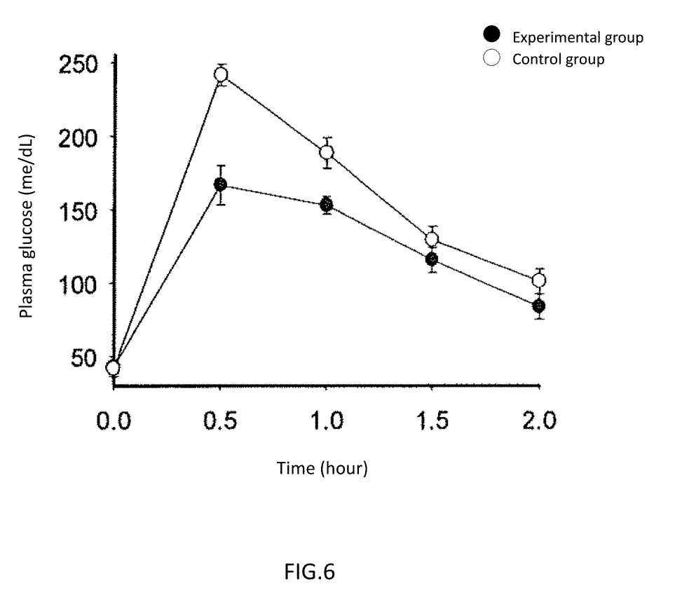

As shown in FIGS. 4 to 6, regardless of the concentration of the extract, it has the efficacy of reducing postprandial blood glucose, and the efficacy of reducing blood sugar is significantly increased in the high dosage group.

### Example 5: Comparison of Inhibitory Effects of Syzygium zeylanicum L. Extract

In this example, an alpha-glucosidase inhibitor known as a Euonymus laxiflorus Champ (abbreviated as ELC), an alpha-glucosidase inhibitor commercially available to provide diabetes patients (acarbose), and the Syzygium zeylanicum L. extract of the present invention (abbreviated as SZL) were subjected to a rat alpha-glucosidase inhibition test.

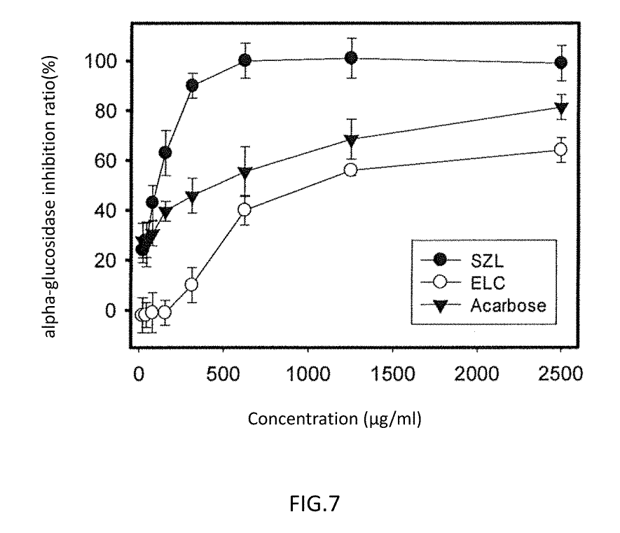

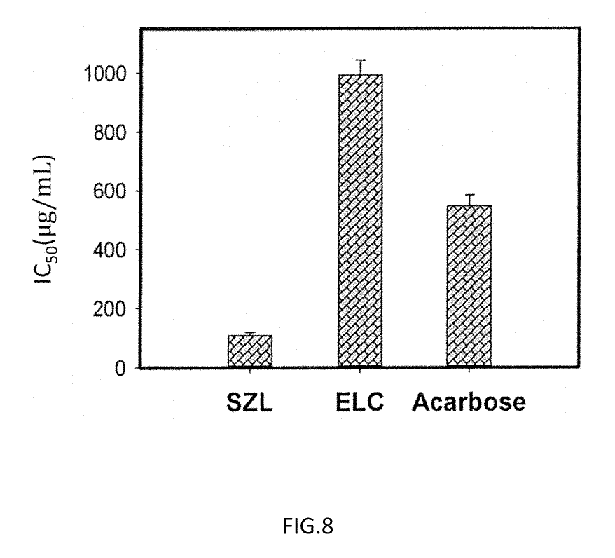

As shown in FIGS. 7 and 8, the Syzygium zeylanicum L. extract of the present invention had higher inhibitory effect of alpha-glucosidase than that ELC and acarbose, regardless of the concentration or IC₅₀. Moreover, the Syzygium zeylanicum L. extract of the present invention had stronger inhibitory activity than acarbose for medical use.

In summary, the Syzygium zeylanicum L. extract of the present invention has a good efficacy of inhibiting alpha-glucosidase and alpha-amylase after oral administration, thereby inhibiting the abnormal increase of postprandial blood glucose in diabetic patients for blood glucose control. The Syzygium zeylanicum L. extract of the present invention can inhibit alpha-amylase, block the absorption of starch, reduce the calorie intake of starch, and further achieve weight loss effect.

1. An application of Syzygium zeylanicum L. extract, characterized in that Syzygium zeylanicum L. extract is used to prepare an orally pharmaceutical for inhibiting alpha-glucosidase.

2. An application of Syzygium zeylanicum L. extract, characterized in that Syzygium zeylanicum L. extract is used to prepare an orally pharmaceutical for inhibiting alpha-amylase.

3. The application of the Syzygium zeylanicum L. extract according to claim 2, wherein the Syzygium zeylanicum L. extract can reduce the absorption of starch by oral inhibition of alpha-amylase, thereby further has weight loss effect.

4. The application of the Syzygium zeylanicum L. extract according to claim 1, wherein the oral dosage of the Syzygium zeylanicum L. extract is from 50 mg/kg to 200 mg/kg.

5. The application of the Syzygium zeylanicum L. extract according to claim 1, wherein the Syzygium zeylanicum L. extract is prepared as following steps:
Step 1: grinding a portion of Syzygium zeylanicum L. at room temperature into a powder;
Step 2: mixing the powder and a solvent at a ratio of 1:10 by volume, and shaking at room temperature for 24 hours, and producing a mixture;
Step 3: filtering the mixture of the step 2, and producing a residue and an extract;
Step 4: mixing the residue of the step 3 and the solvent of the step 2 by 20 times volume of the powder of the step 1, and then shaking at room temperature for 24 hours;
Step 5: repeat at least once of the steps 3 and 4, and collecting the extract;
Step 6: vacuum drying the extract at 60° C. to obtain Syzygium zeylanicum L. extract and storing at −30° C.

6. The application of the Syzygium zeylanicum L. extract according to claim 5, wherein the solvent is selected from the group consisting of ionic water, butanol, ethyl acetate, methanol and ethanol.

7. The application of the Syzygium zeylanicum L. extract according to claim 6, wherein the solvent is ethyl acetate.

8. The application of the Syzygium zeylanicum L. extract according to claim 5, wherein the portion of the Syzygium zeylanicum L. is selected from the group consisting of bark, fruit and leaves.

9. The application of the Syzygium zeylanicum L. extract according to claim 8, wherein the portion of the Syzygium zeylanicum L. is bark.

10. The application of the Syzygium zeylanicum L. extract according to claim 2, wherein the oral dosage of the Syzygium zeylanicum L. extract is from 50 mg/kg to 200 mg/kg.

11. The application of the Syzygium zeylanicum L. extract according to claim 2, wherein the Syzygium zeylanicum L. extract is prepared as following steps:
Step 1: grinding a portion of Syzygium zeylanicum L. at room temperature into a powder;
Step 2: mixing the powder and a solvent at a ratio of 1:10 by volume, and shaking at room temperature for 24 hours, and producing a mixture;
Step 3: filtering the mixture of the step 2, and producing a residue and an extract;
Step 4: mixing the residue of the step 3 and the solvent of the step 2 by 20 times volume of the powder of the step 1, and then shaking at room temperature for 24 hours;
Step 5: repeat at least once of the steps 3 and 4, and collecting the extract;
Step 6: vacuum drying the extract at 60° C. to obtain Syzygium zeylanicum L. extract and storing at −30° C.

12. The application of the Syzygium zeylanicum L. extract according to claim 3, wherein the oral dosage of the Syzygium zeylanicum L. extract is from 50 mg/kg to 200 mg/kg.
